(19) United States
(12) Patent Application Publication
(10) Pub. No.: US 2022/0096665 A1
(43) Pub. Date: Mar. 31, 2022

(54) BIMODAL NANOPARTICLE CONJUGATES FOR NON-INVASIVE CENTRAL NERVOUS SYSTEM TISSUE IMAGING

(71) Applicant: University of Georgia Research Foundation, Inc., Athens, GA (US)

(72) Inventors: Puliyur Seshadri MohanKumar, Athens, GA (US); Sheba M.J. MohanKumar, Athens, GA (US); Yen-jun Chuang, Athens, GA (US)

(73) Assignee: University of Georgia Research Foundation, Inc., Athens, GA (US)

(21) Appl. No.: 17/296,129

(22) PCT Filed: Nov. 20, 2019
(86) PCT No.: PCT/US2019/062438

§ 371 Date: May 21, 2021

Related U.S. Application Data
(60) Provisional application No. 62/770,488, filed on Nov. 21, 2018.

(51) Int. Cl. A61K 49/18; A61K 49/00; A61K 9/00

(52) U.S. Cl. CPC A61K 49/1887; A61K 49/0002; A61K 49/0021; B82Y 5/00; A61K 49/0093; A61K 9/0085; A61K 49/0054

(57) ABSTRACT

Ligand-bimodal nanoparticle conjugates capable of crossing the blood-brain barrier are disclosed. Methods of making and using the conjugates also are disclosed. The bimodal nanoparticle includes a polymeric matrix, one or more magnetic particles disposed within the polymeric matrix or conjugated to an outer surface of the polymeric matrix, and a dye disposed within the polymeric matrix. A ligand for a blood-brain barrier amino acid transporter is conjugated to the outer surface of the bimodal nanoparticle.

### CROSS-REFERENCE TO RELATED APPLICATION

This application claims the benefit of the earlier filing date of U.S. Provisional Application No. 62/770,488, filed Nov. 21, 2018, which is incorporated by reference herein in its entirety.

### FIELD

This invention concerns ligand-bimodal nanoparticle conjugates capable of crossing the blood-brain barrier, as well as methods of making and using the conjugates.

### BACKGROUND

Several contrast agents for imaging the brain at a molecular level have been developed, but very few of them are capable of crossing the blood-brain barrier (BBB). To circumvent the issue of crossing the BBB, methods have been developed to disrupt the BBB to allow imaging agent to enter the brain. However, these methods have not found traction as they cause neuronal injuries. Thus, it is important to develop probes that can cross the BBB to advance neuroimaging.

### SUMMARY

Embodiments of ligand-bimodal nanoparticle conjugates capable of crossing the blood-brain barrier are disclosed. Methods for making and using the conjugates also are disclosed.

In some embodiments, a ligand-bimodal nanoparticle conjugate includes (i) a bimodal nanoparticle comprising a polymeric matrix, one or more magnetic particles disposed within the polymeric matrix or conjugated to an outer surface of the polymeric matrix, and a near-infrared dye disposed within the polymeric matrix; and (ii) a ligand for a blood-brain barrier amino acid transporter, the ligand conjugated to the outer surface of the bimodal nanoparticle. The ligand may comprise an amino acid precursor of a neurotransmitter. In certain embodiments, the ligand comprises levodopa (L-DOPA), 5-hydroxytryptophan (5-HTP), or a combination thereof.

The magnetic particles may comprise a magnetic metal, a compound comprising a magnetic metal, or a combination thereof. In some embodiments, the magnetic particles comprise Fe₃O₄, Fe₂O₃, Fe, Gd, or a combination thereof. In any or all embodiments, the dye may comprise silicon 2,3-naphthalocyanine bis(trihexylsilyloxide) (NIR775).

In any or all embodiments, the polymeric matrix may comprise a semiconducting polymer and an amphiphilic polymer. In some embodiments, the semiconducting polymer is poly[2-methoxy-5-(2-ethylhexyloxy)-1,4-phenylenevinylene](MEH-PPV), poly[2,5-bis(octyloxy)-1,4-phenylenevinylene](BOPPV), poly(5-(2-ethyl hexyl oxy)-2-methoxycyanoterephthalyli dene) (MEHCPV), poly[2-methoxy-5-(2-ethylhexyloxy)-1,4-phenylene]end-capped with dimethylphenyl (MEHPP), poly[2-methoxy-5-(3′,7′-dimethyloctyloxy)-1,4-phenylenevinylene](MDMO-PPV), poly[9,9-dioctylfluorenyl-2,7-diyl)-alt-co-(1,4-benzo-(2,1′,3)-thiadiazole)](PFBT), poly(2,5-dioctyl-1,4-phenylenevinylene) (POPPV), or any combination thereof; and the amphiphilic polymer is polystyrene grafted with carboxyl-group-functionalized ethylene oxide (PS:PEG:COOH), poly(ethylene glycol)-block-polylactide (PEG-PLA), poly(ethylene glycol)-block-poly(lactide-co-glycolide) (PEG-PLGA), poly(ethylene glycol)-block-polyethylene (PEG-PE), poly(ethylene glycol)-block-poly(ε-caprolactone) (PEG-PCL), poly(ethylene glycol)-block-poly(propylene glycol)-block-poly(ethylene glycol) (PEG-PPG-PEG), poly(propylene glycol)-block-poly(ethylene glycol)-block-poly(propylene glycol) (PPG-PEG-PPG), or any combination thereof. In certain embodiments, the polymeric matrix comprises MEH-PPV and PS:PEG:COOH. A weight ratio of MEH-PPV to PS:PEG:COOH may be within a range of from 1.5:1 to 1:1.5.

In any or all embodiments, a diameter of the ligand-bimodal nanoparticle conjugate may be ≤100 nm. In any or all embodiments, the ligand-bimodal nanoparticle conjugate may have a ligand:bimodal nanoparticle weight ratio of from 0.5:1 to 10:1.

Embodiments of a method for imaging central nervous system tissue in a subject include administering a plurality of the disclosed ligand-bimodal nanoparticle conjugates to a subject and subsequently imaging central nervous system tissue in the subject. Suitable imaging techniques include magnetic resonance imaging, fluorescence resonance energy transfer imaging, or a combination thereof. In some embodiments, the subject is a human.

In one embodiment, the ligand-bimodal nanoparticle conjugates comprise L-DOPA, and imaging the central nervous system tissue in the subject visualizes dopaminergic neurons, noradrenergic neurons, or both dopaminergic and noradrenergic neurons bound by the ligand-bimodal nanoparticle conjugates. In another embodiment, the ligand-bimodal nanoparticle conjugates comprise 5-HTP, and imaging the central nervous system tissue in the subject visualizes serotonergic neurons bound by the ligand-bimodal nanoparticle conjugates.

In any or all embodiments, administering the ligand-bimodal nanoparticle conjugates may include intravenously injecting the ligand-bimodal nanoparticle conjugates or a pharmaceutical composition comprising the ligand-bimodal nanoparticle conjugates. In some embodiments, from 100-500 μg of the ligand-bimodal nanoparticle conjugates per kilogram body weight are administered to the subject. In any or all embodiments, imaging may be performed from one hour to ten days after administering the plurality of ligand-bimodal nanoparticle conjugates to the subject.

The foregoing and other objects, features, and advantages of the invention will become more apparent from the following detailed description, which proceeds with reference to the accompanying figures.

### BRIEF DESCRIPTION OF THE DRAWINGS

FIG. 1 is a schematic diagram illustrating one synthetic route for preparing bimodal nanoparticles (MPdots) as disclosed herein.

FIG. 2 is a transmission electron microscope (TEM) image of MPdots prepared as disclosed herein.

FIG. 3 shows the magnetic properties (T2 maps) of MPdots of varying concentrations−0.2 mL of 0.1, 1, 5, 10, 15 and 20 μg/mL MPdot solutions.

FIG. 4 shows normalized UV-Vis absorbance and fluorescent emission spectra of MPdots.

FIG. 5 shows near-infrared afterglow images of MPdots at concentrations of 0, 0.2, 0.5, 1, 10, 20, 50 μg/mL.

FIG. 6 is a graph showing the total flux of MPdots as a function of concentration.

FIG. 7 shows the results of a cell toxicity assay with PC12 cells and varying concentrations (0-50 μg/mL) of MPdots.

FIG. 8 shows the results of a cell toxicity assay with neural progenitor cells and varying concentrations (0-50 μg/mL) of MPdots.

FIG. 9 is a graph illustrating cell viability with PC12 cells incubated with varying concentrations (0-100 μg/mL) of MPdots.

FIG. 10 shows images of in vivo T2* mapping of Sprague Dawley rats three days after intracerebroventricular (ICV) injections of PBS (vehicle), MPdots without L-DOPA (MPdots) or MPdots conjugated to L-DOPA (L-DOPA).

FIGS. 11A-11H are images of control rat brain tissue sections (FIGS. 11A-D) and brain tissue sections labeled with L-DOPA-MPdot conjugates (FIGS. 11E-H). FIGS. 11A and 11E are bright-field images; FIGS. 11B and 11F are green fluorescent images; FIGS. 11C and 11G are NIR fluorescence images; FIGS. 11D and 11H are merged images.

FIGS. 12A-12F are MRI images of rat brain tissue following intravenous injection with PBS (FIGS. 12A, 12C, 12E) or L-DOPA-MPdot conjugates (FIGS. 12B, 12D, 12F). FIGS. 12A and 12B are qualitative T2-weighted images; FIGS. 12C and 12D are quantitative T2 maps (the gray scale units are milliseconds); FIGS. 12E and 12F are quantitative T2 maps with a pseudo color scale (the color scale units are milliseconds).

FIGS. 13A-13C are graphs showing T2 map data over time for rat brain tissue following intravenous injection with PBS or L-DOPA-MPdot conjugates. Results are shown for the arcuate nucleus (FIG. 13A), zona incerta (FIG. 13B), and substantia nigra (FIG. 13C).

FIG. 14 shows images of rat hindbrain tissue following intravenous injection with PBS or L-DOPA-MPdot conjugates.

FIG. 15 shows images of rat arcuate nucleus tissue following intravenous injection with L-DOPA-MPdot conjugates (L-DOPA), unconjugated MPdots (MPdot), or vehicle (PBS); the upper images show the raw data converted to green color scale, and the lower images show the raw data converted to a 16-color scale. However, the images are provided in grayscale.

### DETAILED DESCRIPTION

This disclosure concerns embodiments of ligand-bimodal nanoparticle conjugates. In some embodiments, the ligand-bimodal nanoparticle conjugates are capable of crossing the blood-brain barrier and can be used for non-invasive imaging of central nervous system tissue. Methods of making and using the conjugates are also disclosed.

The blood-brain barrier (BBB) is a unique membranous barrier that tightly segregates the brain from the circulating blood. This barrier, formed by special endothelial cells sealed with tight junctions and a complete absence of pinocytic activity, restricts the molecular exchange to transcellular transport. Only unionized, lipophilic, and low molecular weight molecules can diffuse freely through the endothelial membrane and may passively cross the BBB. Polar molecules and small ions are totally excluded. Other essential compounds such as amino acids, hexoses, neuropeptides, and proteins need specific carriers or transport systems to permeate the brain. Failure of contrast agents to cross the BBB in appreciable quantity may be attributed to their uptake by the reticulo-endothelial system (RES). Consequently, imaging of structure and function in the brain is greatly limited by the ability to deliver contrast agents with molecular specificity across the BBB.

To achieve non-invasive central nervous system tissue imaging, it is preferable that contrast agents are (i) nontoxic, biodegradable, and biocompatible, (ii) less than 100 nm in diameter, (iii) physically stable in vivo and in vitro, (iv) capable of avoiding non-specific uptake by the RES to extend blood circulation time, (v) deliverable via receptor-mediated transcytosis across brain capillary endothelial cells, and/or (vi) scalable and cost-effective to manufacture. Embodiments of the disclosed ligand-bimodal nanoparticle conjugates possess some or all of these qualities.

Different modalities of imaging techniques can be complementary. Advantageously, the disclosed bimodal nanoparticles and conjugates thereof can be simultaneously detected by optical imaging and MRI as they incorporate a luminescent core and paramagnetic ions (which generate MRI signals) into the same particle. In some embodiments, the bimodal nanoparticles combine the advantages of high sensitivity (e.g., from optical detection) with the potential of true three-dimensional imaging of biological nanostructures and processes at cellular resolution (e.g., from MRI detection). In some embodiments, presence of the disclosed bimodal nanoparticles and conjugates thereof in any specific area of the brain or other organs of the body can be determined using an immunohistochemistry protocol to visualize the bimodal nanoparticles in three dimensions using light-sheet microscopy without the need for antibodies.

### I. Definitions and Abbreviations

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

Definitions of common terms in chemistry may be found in Richard J. Lewis, Sr. (ed.), Hawley's Condensed Chemical Dictionary, published by John Wiley & Sons, Inc., 2016 (ISBN 978-1-118-13515-0).

In order to facilitate review of the various embodiments of the disclosure, the following explanations of specific terms are provided:

Amino acid: An organic acid containing both a basic amino group (—NH₂) and an acidic carboxyl group (—COOH).

Amino acid precursor of a neurotransmitter: A modified amino acid, which binds to an amino acid transporter and is transported across the blood-brain barrier. The amino acid precursor binds and is internalized into neurons in the central nervous system.

Amino acid transporter: A membrane protein capable of transporting amino acids across the membrane.

Bimodal: Having or involving two modes. As used herein, the term “bimodal” refers to having two modes of detection, e.g., magnetic detection and near-infrared light emission detection.

Blood-brain barrier (BBB): A selective semipermeable border formed by endothelial cells lining the central nervous system microvasculature and allowing only certain agents circulating in the blood to pass through the barrier and contact central nervous system tissue.

Central nervous system tissue: In vertebrates, central nervous system tissue includes the tissues of the brain and spinal cord.

Conjugate: Two or more moieties directly or indirectly coupled together. For example, a first moiety may be covalently or noncovalently (e.g., electrostatically) coupled to a second moiety. As used herein, the term “conjugate” refers to a ligand coupled to an outer surface of a bimodal nanoparticle.

Conjugating, joining, bonding or linking: Coupling a first moiety to a second moiety. This includes, but is not limited to, covalently bonding one molecule to another molecule, such as covalently bonding a ligand to an outer surface of a bimodal nanoparticle.

Dopaminergic neurons: Neurons that synthesize the neutransmitter dopamine and downstream neurotransmitters, such as norepinephrine. Dopaminergic neurons are primarily located in the midbrain and are the main source of the neurotransmitter dopamine in the mammalian central nervous system. Dopaminergic neurons are involved in the control of multiple brain functions including voluntary movement and a broad array of behavioral processes such as mood, reward, addiction, and stress.

Emission or emission signal: The light of a particular wavelength generated from a source. In particular examples, an emission signal is emitted from a near-infrared dye after the near-infrared dye absorbs light at its excitation wavelength(s).

Excipient: A physiologically inert substance that is used as an additive in a pharmaceutical composition. As used herein, an excipient may be incorporated within particles of a pharmaceutical composition, or it may be physically mixed with particles of a pharmaceutical composition. An excipient can be used, for example, to dilute an active agent and/or to modify properties of a pharmaceutical composition. Examples of excipients include but are not limited to polyvinylpyrrolidone (PVP), tocopheryl polyethylene glycol 1000 succinate (also known as vitamin E TPGS, or TPGS), dipalmitoyl phosphatidyl choline (DPPC), trehalose, sodium bicarbonate, glycine, sodium citrate, and lactose.

Excitation or excitation signal: The light of a particular wavelength necessary and/or sufficient to excite an electron transition to a higher energy level. In particular examples, an excitation is the light of a particular wavelength necessary and/or sufficient to excite a fluorophore to a state such that the fluorophore will emit a different (such as a longer) wavelength of light than the wavelength of light from the excitation signal.

5-HTP: 5-hydroxytryptophan. An amino acid precursor of serotonin.

L-DOPA: Levodopa, L-3,4-dihydroxyphenylalanine. An amino acid precursor of dopamine, norepinephrine, and epinephrine.

Ligand: A molecule that binds to a receptor, having a biological effect.

Magnetic: Exhibiting magnetism, e.g., attracted by magnetic fields. The term “ferromagnetic” refers to materials that are susceptible to magnetization by exposure to an applied magnetic field, which may persist after removal of the applied field.

Nanoparticle: A nanoscale particle with a size that is measured in nanometers, for example, a nanoscopic particle that has at least one dimension of less than about 100 nm.

Near infrared (NIR): A region of the electromagnetic spectrum between the visible region and the infrared region. There is no set definition for the boundaries of the near-infrared region, but definitions include the wavelength ranges from 650-2500 nm, 750-2500 nm, 780-2500 nm, 800-2500 nm, 700-1400 nm, or 780-3000 nm. As used herein, NIR refers to the wavelength region of 650-2500 nm.

NIR775: Silicon 2,3-naphthalocyanine bis(trihexylsilyloxide), a near-infrared dye.

Noradrenergic neurons: Neurons that synthesize norepinephrine. Noradrenergic neurons are involved in alertness, arousal, and readiness for action, e.g., the “fight-or-flight” response. Noradrenergic neurons are located in the caudal ventrolateral part of the medulla, the solitary nucleus of the brainstem, the locus coeruleus of the brain, and the spinal cord.

Number Average Molecular Weight (Mn): The number average molecular weight of a polymer is the total weight of all polymer molecules in a sample divided by the total number of polymer molecules in the sample.

Particle: The term “particle” is commonly understood to mean a very small or tiny mass of a material. As used herein, the term particle may refer to a magnetic particle having a size within a range of from 1 to 10 nm.

Pharmaceutically acceptable carrier: The pharmaceutically acceptable carriers (vehicles) useful in this disclosure are conventional. Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of one or more therapeutic compositions and additional pharmaceutical agents. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In some examples, the pharmaceutically acceptable carrier may be sterile to be suitable for administration to a subject (for example, by parenteral, intramuscular, or subcutaneous injection). In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. In some examples, the pharmaceutically acceptable carrier is a non-naturally occurring or synthetic carrier. The carrier also can be formulated in a unit-dosage form that carries a preselected therapeutic dosage of the active agent, for example in a pill, vial, bottle, or syringe.

Polymer: A molecule of repeating structural units (e.g., monomers) formed via a chemical reaction, i.e., polymerization.

Polymeric matrix: As used herein, the term “polymeric matrix” refers to a polymeric material in which magnetic particles and/or near-infrared dyes are mixed or dispersed.

Precursor: An intermediate compound. A precursor participates in a chemical reaction to form another compound.

Serotonergic neurons: Neurons that synthesize the neurotransmitter serotonin. Serotonergic neurons primarily located in the Raphe Nuclei found in the medulla, pons, and midbrain.

Subject: An animal (human or non-human) subjected to a treatment, observation or experiment.

### II. Bimodal Nanoparticles and Conjugates

Embodiments of bimodal nanoparticles (MPdots) and conjugates thereof are disclosed. Advantageously, some embodiments of the disclosed MPdots and conjugates are nontoxic, biodegradable, biocompatible, less than 100 nm in diameter, and/or cost effective to produce. Embodiments of the disclosed ligand-bimodal nanoparticles conjugates are based on an organic nanoparticle that incorporates a magnetic particle and a NIR dye, and are tailored to cross the blood-brain barrier and enable non-invasive central nervous system tissue imaging. The nanoparticle is bimodal and can be used for magnetic resonance imaging (MRI) as well as near infrared imaging.

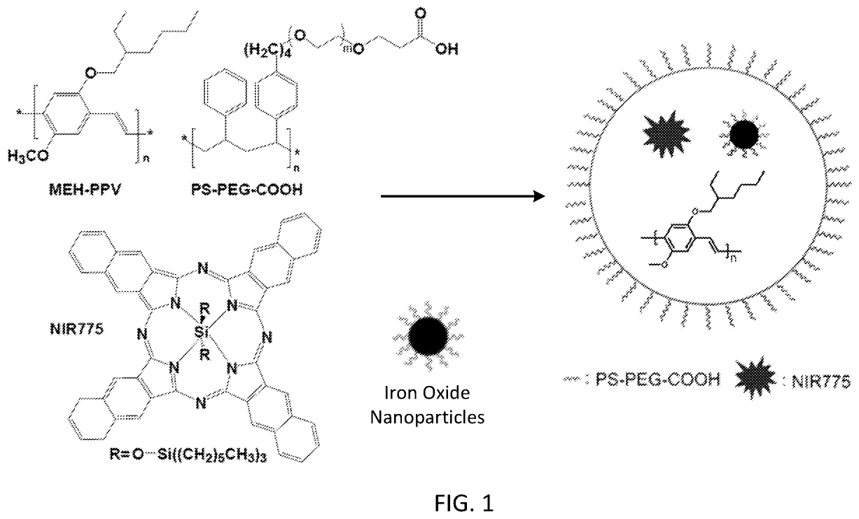

Embodiments of the disclosed MPdots comprise a polymeric matrix, one or more magnetic particles disposed within the polymeric matrix or conjugated to an outer surface of the polymeric matrix, and a near-infrared dye disposed within the polymeric matrix (FIG. 1). A ligand for a blood-brain barrier amino acid transporter is conjugated to an outer surface of the bimodal nanoparticle to provide a ligand-bimodal nanoparticle conjugate.

In some embodiments, the polymeric matrix comprises a semiconducting polymer and an amphiphilic polymer. Exemplary semiconducting polymers include, but are not limited to, poly[2-methoxy-5-(2-ethylhexyloxy)-1,4-phenylenevinylene](MEH-PPV), poly[2,5-bis(octyloxy)-1,4-phenylenevinylene](BOPPV), (2-ethylhexyloxy)-2-methoxycyanoterephthalylidene) (MEHCPV), poly[2-methoxy-5-(2-ethylhexyloxy)-1,4-phenylene]end-capped with dimethylphenyl (MEHPP), poly[2-methoxy-5-(3′,7′-dimethyloctyloxy)-1,4-phenylenevinylene](MDMO-PPV), poly[9,9-dioctylfluorenyl-2,7-diyl)-alt-co-(1,4-benzo-(2,1′,3)-thiadiazole)](PFBT), poly(2,5-dioctyl-1,4-phenylenevinylene) (POPPV), and combinations thereof. Exemplary amphiphilic polymers include diblock copolymers (such as polystyrene grafted with carboxyl-group-functionalized ethylene oxide (PS:PEG:COOH), poly(ethylene glycol)-block-polylactide (PEG-PLA), poly(ethylene glycol)-block-poly(lactide-co-glycolide) (PEG-PLGA), poly(ethylene glycol)-block-polyethylene (PEG-PE), and poly(ethylene glycol)-block-poly(ε-caprolactone) (PEG-PCL)), triblock copolymers (such as poly(ethylene glycol)-block-poly(propylene glycol)-block-poly(ethylene glycol) (PEG-PPG-PEG), and poly(propylene glycol)-block-poly(ethylene glycol)-block-poly(propylene glycol) (PPG-PEG-PPG)), and combinations thereof.

In certain embodiments, the polymeric matrix comprises MEH-PPV and PS:PEG_COOH.

In some embodiments, the polymeric matrix comprises MEH-PPV and PS:PEG:COOH in a weight ratio of from 1.5:1 to 1:1.5. Changes in the weight ratios may change the optical properties of the MPdot. In certain embodiments, the polymeric matrix includes a 1:1 ratio, on a weight basis, of MEH-PPV to PS:PEG:COOH. In some embodiments, the MEH-PPV has a number average molecular weight (Mn) of 40-70 kDa. In some embodiments, the PS:PEG:COOH includes PS with Mn=20-25 kDa and PEG-COOH with Mn=1-1.5 kDa, providing the polymer with a total molecular weight of 21-26.5 kDa. In certain embodiments, the PS has Mn=21.7 kDa and PEG-COOH has Mn=1.2 kDa. The PEG molecular weight affects the ability to keep the MPdots in circulation for prolonged periods of time. A PEG molecular weight less than 1 kDa is ineffective to provide prolonged circulation. Additionally, the overall molecular weight of the PS:PEG:COOH polymer affects the optical properties (fluorescence and afterglow) with increasing molecular weight lowering the efficacy.

In some embodiments, the polymeric matrix forms nanoparticles wherein the MEH-PPV is preferentially localized to an interior portion of the nanoparticle with PS:PEG:COOH molecules preferentially located on the nanoparticle surface (FIG. 1). Without wishing to be bound by a particular theory of operation, the PEG portions may form a brush-like PEG layer on the outer surface, thereby reducing adsorption of proteins from human plasma. The low protein adsorption, coupled with a low level of complement activation, may prevent uptake of the nanoparticles by the mononuclear phagocytic system in vivo.

The disclosed MPdots include one or more magnetic particles disposed within the polymeric matrix. In some embodiments, the magnetic particles comprise a magnetic metal, a compound comprising a magnetic metal, or a combination thereof. Suitable magnetic particles include, but are not limited to, magnetic particles comprising Fe₃O₄, Fe₂O₃, Fe, Gd, or a combination thereof. In some embodiments, the magnetic particles are nontoxic when administered to a subject in the form of the ligand-bimodal nanoparticle conjugates. In certain embodiments, the magnetic particles comprise Fe₃O₄. In any or all embodiments, the magnetic particles may have an average size of from 1 to 10 nm. In some embodiments, the bimodal nanoparticles comprise from 5-25 wt %, such as 10-20 wt % or 12-16 wt % magnetic particles. Alternatively, the disclosed MPdots may include a hydrophilic contrast agent, e.g., a hydrophilic gadolinium-based contrast agent, conjugated to the outer surface of the MPdots.

The disclosed MPdots further include a near-infrared (NIR) dye or a visible emission dye disposed within the polymeric matrix. In some embodiments, the MPdots include silicon 2,3-naphthalocyanine bis(trihexylsilyloxide) (NIR775), an NIR dye. In some embodiments, the bimodal nanoparticles comprise from 0.05-5 wt % of the dye, such as 0.1-2 wt %, 0.1-1 wt %, or 0.4-0.6 wt % of the dye.

In certain embodiments, the MPdots comprise a polymeric matrix comprising MEH-PPV and PS:PEG:COOH, NIR775 dye, and Fe₃O₄. The Fe₃O₄ particles can be visualized by magnetic resonance imaging, and the NIR775 dye can be visualized by fluorescence resonance energy transfer (FRET) imaging. In FRET imaging, the MPdots are excited with green light, whereby the MPdot emits red light from the MEH-PPV. The emission energy is transferred to the NIR775 dye by the FRET mechanism to provide a NIR emission. Advantageously, the NIR emission is far away from the green excitation light (in terms of the wavelength), which reduces light interference from excitation. The PS moiety of the PS:PEG:COOH polymer forms a core that entraps MEH-PPV and NIR775. The PEG moiety is located at the MPdot surface, and reduces or prevents MPdot interactions with serum proteins, thereby prolonging circulation of the MPdot in a subject.

In any or all embodiments, the MPdots may have a total molecular weight (polymers, magnetic particles, and dye)≤350 kDa, such as ≤200 kDa. In some embodiments, the total molecular weight is within a range of from 22-350 kDa, such as 22-200 kDa, 50-200 kDa, or 100-200 kDa.

A ligand-bimodal nanoparticle conjugate comprises an MPdot as disclosed herein and a ligand for a blood-brain barrier amino acid transporter, wherein the ligand is conjugated to an outer surface of the MPdot. Each conjugate may include more than one ligand conjugated to the outer surface of the MPdot. In some embodiments, the ligand comprises an amino acid precursor of a neurotransmitter. Exemplary ligands include, but are not limited to, levodopa (L-DOPA), 5-hydroxytryptophan (5-HTP), or a combination thereof. In certain embodiments, the ligand is L-DOPA L-tyrosine is converted to L-DOPA by the enzyme tyrosine hydroxylase. L-DOPA is preferred over L-tyrosine as a ligand for two primary reasons. High doses of L-tyrosine may cause unwanted side effects in a subject, such as diarrhea, nausea, vomiting, headaches, and/or insomnia. Additionally, coadministration of L-tyrosine and a monoamine oxidase inhibitor (e.g., antidepressants such as isocarboxazid, phenelzine, tranylcypromine, and selegiline) will increase the subject's blood pressure. L-DOPA has the same benefit as L-tyrosine of crossing the blood-brain barrier, but does not induce or minimizes the side effects associated with L-tyrosine.

In any or all embodiments, the conjugate may include a weight ratio of ligands to MPdots within a range of 1-10, such as a weight ratio within a range of 1-5, 1-3, or 1-2. When a weight ratio of ligands to MPdots is greater than 10, aggregation of the MPdots may occur. Conversely, when the weight ratio is too low, there may insufficient conjugation and reduced efficacy in crossing the blood-brain barrier. A maximum theoretical concentration of L-DOPA can be estimated by the size of the nanoparticles and is calculated to be ˜2.17×10⁻²⁰ mol/unit nanoparticle.

In some embodiments, the ligand-bimodal nanoparticle conjugates have an average diameter of ≤100 nm, such as an average diameter of 10-100 nm, or 10-50 nm.

In one embodiment a ligand-bimodal nanoparticle conjugate comprises (i) a bimodal nanoparticle comprising a polymeric matrix comprising MEH-PPV and PS:PEG:COOH, one of more Fe₃O₄ particles disposed within the polymeric matrix, NIR775 dye disposed within the polymeric matrix, and (ii) one or more L-DOPA ligands conjugated to an outer surface of the bimodal nanoparticle. In another embodiment, a ligand-bimodal nanoparticle conjugate comprises (i) a bimodal nanoparticle comprising a polymeric matrix comprising MEH-PPV and PS:PEG:COOH, one of more Fe₃O₄ particles disposed within the polymeric matrix, NIR775 dye disposed within the polymeric matrix, and (ii) one or more 5-HTP ligands conjugated to an outer surface of the bimodal nanoparticle.

This disclosure also encompasses pharmaceutical compositions comprising a ligand-bimodal nanoparticle conjugate as disclosed herein, and a pharmaceutically acceptable carrier. Pharmaceutical compositions comprising embodiments of the disclosed conjugates may comprise a single conjugate composition or may comprise plural conjugate compositions (e.g., L-DOPA conjugates and 5-HTP conjugates, or conjugates with differing magnetic particles and/or differing IR dyes).

Advantageously, the pharmaceutical composition includes an amount of the ligand-bimodal nanoparticle conjugate effective for in vivo imaging, e.g., by MRI or fluorescence imaging techniques. In some non-limiting examples, the pharmaceutical composition may include from 0.1-10 mg/mL of the conjugates in a pharmaceutically acceptable carrier, such as 1-10 mg/mL or 2-5 mg/mL of the conjugates in the pharmaceutically acceptable carrier. In some embodiments, an effective amount of the conjugates for imaging is within a range of 100-500 μg/kg body weight, such as 100-300 μg/kg body weight. In certain examples, 50 μg conjugates per 250 g body mass was effective in rats.

Pharmaceutical compositions for administration to a subject can include carriers, thickeners, diluents, buffers, preservatives, surface active agents and the like. Pharmaceutical compositions can also include one or more additional active ingredients such as anti-inflammatory agents, anesthetics, and the like. The pharmaceutical compositions may be manufactured by conventional methods known to those of ordinary skill in the art.

The pharmaceutically acceptable carriers useful for these formulations are conventional. Remington: The Science and Practice of Pharmacy, by E. W. Martin, Mack Publishing Co., Easton, Pa., 22nd Edition (2012), describes compositions and formulations suitable for pharmaceutical delivery of the conjugates disclosed herein. In general, the nature of the carrier will depend on the particular mode of administration being employed. For instance, parenteral formulations usually contain injectable fluids that include pharmaceutically and physiologically acceptable fluids such as water, physiological saline, balanced salt solutions, aqueous dextrose, glycerol or the like as a vehicle.

Useful injectable preparations include sterile suspensions or emulsions of the conjugate(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use.

### III. Method of Making Bimodal Nanoparticles and Conjugates

Embodiments of the disclosed bimodal nanoparticles, or MPdots, may be prepared by any suitable means, such as by nanoprecipitation as illustrated in FIG. 1. Briefly, stock solutions of the polymers, magnetic particles, and NIR dye in a suitable solvent are prepared. Suitable solvents include polar organic solvents such as tetrahydrofuran. Desired quantities of the stock solutions are added to water with constant mixing, e.g., sonication. The organic solvent is subsequently removed, e.g., by evaporation. Unreacted components are removed by ultrafiltration, and the bimodal nanoparticles may be washed with water and collected by centrifugation.

The isolated bimodal nanoparticles are conjugated to a ligand by conventional methods. In some embodiments, L-DOPA is conjugated to the carboxyl groups on the MPdots using N-ethyl-N′-(3-(dimethylamino propyl) carbodiimide and N-hydroxysuccinimide. In some embodiments, 5-HTP is conjugated to the MPdots via its amino, carboxyl, or hydroxyl groups. Unreacted components are removed by ultrafiltration, and the conjugates may be washed with water and collected by centrifugation.

### IV. Methods of Use

Embodiments of the disclosed ligand-bimodal nanoparticle conjugates are useful for imaging central nervous system tissue. Imaging may be performed in vivo or ex vivo (e.g., in a biological sample obtained from a subject). In some embodiments, the ligand-bimodal nanoparticle conjugates are used for non-invasive imaging of central nervous system tissue. In certain embodiments, imaging can be used to detect neurochemical changes. In one embodiment, the central nervous system tissue is brain tissue. In another embodiment, the central nervous system is spinal cord tissue.

In some embodiments, a plurality of ligand-bimodal nanoparticle conjugates are administered to a subject, and subsequent imaging of central nervous system tissue in the subject is performed. In some embodiments, the subject is a human.

In any or all embodiments, administering the ligand-bimodal nanoparticle conjugates may comprise intravenously injecting the ligand-bimodal nanoparticle conjugates or a pharmaceutical composition comprising the ligand-bimodal nanoparticle conjugates. In some embodiments, the subject may be administered from 1-1000 μg/kg body weight of the ligand-bimodal nanoparticle conjugates, such as 100-1000 μg/kg, 100-500 μg/kg, or 100-300 μg/kg body weight.

Imaging may be performed by any method suitable for imaging magnetic particles and/or near-infrared dyes in vivo. In some embodiments, imaging comprises magnetic resonance imaging, fluorescence resonance energy transfer (FRET) imaging, or a combination thereof. Imaging is performed after sufficient time has elapsed to allow transport of the ligand-bimodal nanoparticle conjugates across the blood-brain barrier. In some embodiments, the sufficient time is at least one hour. In certain embodiments, initial imaging is performed from one hour to ten days, such as from one hour to 72 hours, after administering the plurality of ligand-bimodal nanoparticle conjugates to the subject. Subsequent imaging (e.g., to provide a comparison of ongoing neurochemical changes) may be performed several hours or days after the initial imaging. For example, initial imaging may be performed from 1-72 hours after administration, and subsequent imaging may be performed 3-10 days after initial imaging.

In one embodiment, the ligand-bimodal nanoparticle conjugates comprise L-DOPA, and imaging the central nervous system tissue in the subject visualizes dopaminergic and/or noradrenergic neurons bound by the ligand-bimodal nanoparticle conjugates. In another embodiment, the ligand-bimodal nanoparticle conjugates comprise 5-HTP, and imaging the central nervous system tissue in the subject visualizes serotonergic neurons bound by the ligand-bimodal nanoparticle conjugates.

### V. Representative Embodiments

Certain representative embodiments are exemplified in the following numbered clauses.

1. A ligand-bimodal nanoparticle conjugate, comprising: a bimodal nanoparticle comprising a polymeric matrix, one or more magnetic particles disposed within the polymeric matrix or conjugated to an outer surface of the polymeric matrix, and a near-infrared dye disposed within the polymeric matrix; and a ligand for a blood-brain barrier amino acid transporter, the ligand conjugated to the outer surface of the bimodal nanoparticle.

2. The ligand-bimodal nanoparticle conjugate of clause 1, wherein the ligand comprises an amino acid precursor of a neurotransmitter. 3. The ligand-bimodal nanoparticle conjugate of clause 1 or clause 2, wherein the ligand comprises levodopa (L-DOPA), 5-hydroxytryptophan (5-HTP), or a combination thereof.

4. The ligand-bimodal nanoparticle conjugate of any one of clauses 1-3, wherein the one or more magnetic particles comprises a magnetic metal, a compound comprising a magnetic metal, or a combination thereof.

5. The ligand-bimodal nanoparticle conjugate of clause 4, wherein the one or more magnetic particles comprises Fe₃O₄, Fe₂O₃, Fe, Gd, or a combination thereof.

6. The ligand-bimodal nanoparticle conjugate of any one of clauses 1-5, wherein the near-infrared dye comprises silicon 2,3-naphthalocyanine bis(trihexylsilyloxide) (NIR775).

7. The ligand-bimodal nanoparticle conjugate of any one of clauses 1-6, wherein the polymeric matrix comprises a semiconducting polymer and an amphiphilic polymer.

8. The ligand-bimodal nanoparticle conjugate of clause 7, wherein the polymeric matrix comprises: poly[2-methoxy-5-(2-ethylhexyloxy)-1,4-phenylenevinylene](MEH-PPV), poly[2,5-bis(octyloxy)-1,4-phenylenevinylene](BOPPV), poly(5-(2-ethylhexyloxy)-2-methoxycyanoterephthalylidene) (MEHCPV), poly[2-methoxy-5-(2-ethylhexyloxy)-1,4-phenylene]end-capped with dimethylphenyl (MEHPP), poly[2-methoxy-5-(3′,7′-dimethyloctyloxy)-1,4-phenylenevinylene](MDMO-PPV), poly[9,9-dioctylfluorenyl-2,7-diyl)-alt-co-(1,4-benzo-(2,1′,3)-thiadiazole)](PFBT), poly(2,5-dioctyl-1,4-phenylenevinylene) (POPPV), or any combination thereof; and polystyrene grafted with carboxyl-group-functionalized ethylene oxide (PS:PEG:COOH), poly(ethylene glycol)-block-polylactide (PEG-PLA), poly(ethylene glycol)-block-poly(lactide-co-glycolide) (PEG-PLGA), poly(ethylene glycol)-block-polyethylene (PEG-PE), poly(ethylene glycol)-block-poly(ε-caprolactone) (PEG-PCL), poly(ethylene glycol)-block-poly(propylene glycol)-block-poly(ethylene glycol) (PEG-PPG-PEG), poly(propylene glycol)-block-poly(ethylene glycol)-block-poly(propylene glycol) (PPG-PEG-PPG), or any combination thereof.

9. The ligand-bimodal nanoparticle conjugate of clause 8, wherein the polymeric matrix comprises MEH-PPV and PS:PEG:COOH.

10. The ligand-bimodal nanoparticle conjugate of clause 9, wherein the polymeric matrix comprises MEH-PPV and PS:PEG:COOH in a weight ratio of from 1.5:1 to 1:1.5.

11. The ligand-bimodal nanoparticle conjugate of any one of clauses 1-10, wherein a diameter of the ligand-bimodal nanoparticle conjugate is ≤100 nm.

12. The ligand-bimodal nanoparticle conjugate of any one of clauses 1-11, wherein the ligand-bimodal nanoparticle conjugate has a ligand:bimodal nanoparticle weight ratio of from 0.5:1 to 10:1.

13. The ligand-bimodal nanoparticle conjugate of any one of clauses 1-12, comprising: a bimodal nanoparticle comprising a polymeric matrix comprising MEH-PPV and PS:PEG:COOH, one or more Fe₃O₄ particles disposed within the polymeric matrix, and NIR775 disposed within the polymeric matrix; and L-DOPA conjugated to an outer surface of the bimodal nanoparticle.

14. A pharmaceutical composition comprising: a plurality of ligand-bimodal nanoparticle conjugates according to any one of clauses 1-13; and a pharmaceutically acceptable carrier.

15. A method, comprising: administering a plurality of ligand-bimodal nanoparticle conjugates according to any one of clauses 1-13 to a subject; and subsequently imaging central nervous system tissue in the subject.

16. The method of clause 15, wherein imaging the central nervous system tissue in the subject comprises magnetic resonance imaging, fluorescence resonance energy transfer imaging, or a combination thereof.

17. The method of clause 15 or clause 16, wherein the ligand-bimodal nanoparticle conjugates comprise L-DOPA, and imaging the central nervous system tissue in the subject visualizes dopaminergic neurons, noradrenergic neurons, or both dopaminergic and noradrenergic neurons bound by the ligand-bimodal nanoparticle conjugates.

18. The method of clause 15 or clause 16, wherein the ligand-bimodal nanoparticle conjugates comprise 5-HTP, and imaging the central nervous system tissue in the subject visualizes serotonergic neurons bound by the ligand-bimodal nanoparticle conjugates.

19. The method of any one of clauses 15-18, wherein administering the ligand-bimodal nanoparticle conjugates comprises intravenously injecting the ligand-bimodal nanoparticle conjugates or a pharmaceutical composition comprising the ligand-bimodal nanoparticle conjugates.

20. The method of any one of clauses 15-19, wherein the subject is a human. 21. The method of any one of clauses 15-20, wherein administering the ligand-bimodal nanoparticle conjugates comprises administering from 100-500 μg of the ligand-bimodal nanoparticle conjugates per kilogram body weight to the subject.

22. The method of any one of clauses 15-21, wherein imaging is performed from one hour to ten days after administering the plurality of ligand-bimodal nanoparticle conjugates to the subject.

### VI. Working Examples

### Example 1

### Preparation and Characterization of Ligand-Bimodal Nanoparticle Conjugates

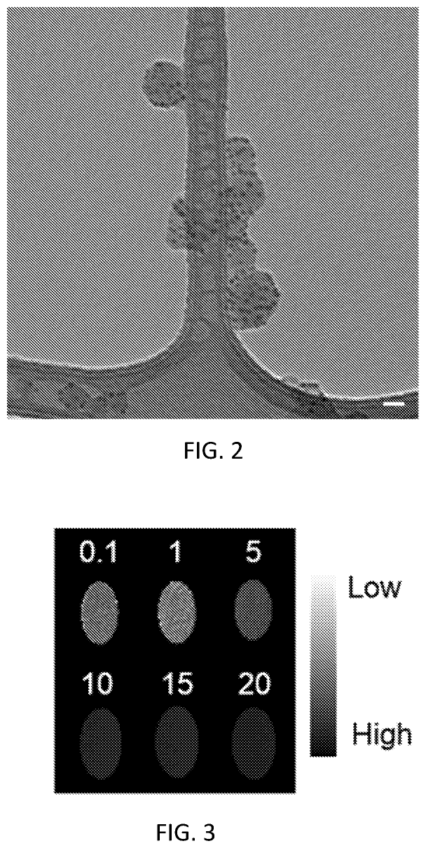

Bimodal nanoparticles (magnetic polymer dots, MPdots) were prepared using a nanoprecipitation technique as illustrated in FIG. 1. Stock solutions of 1 mg/mL poly[2-methoxy-5-(2-ethylhexyloxy)-1,4-phenylenevinylene] (MEH-PPV), polystyrene grafted with carboxyl-group-functionalized ethylene oxide (PS:PEG:COOH), silicon 2,3-naphthalocyanine bis(trihexylsilyloxide) (NIR775), a near infrared dye and iron oxide (Fe₃O₄) were prepared in tetrahydrofuran (THF). The stock solutions (250 μL MEH-PPV, 250 μL PS:PEG:COOH, 3 μL dye NIR775 and 80 μL Fe₃O₄) were mixed in 4 mL of THF. The mixture was added stepwise to 10 mL of biological grade distilled water under constant sonication using a water sonicator (Kontes, NJ), then shaken and sonicated for an additional two minutes. THF was removed by heating on an 80° C. hot plate with continuous bubbling of an argon stream through the solution for 60 min. Unreacted monomers and dyes were removed using a 100 K Amicon® Ultra filter (Millipore), washing with biological grade distilled water and centrifuging at 1000×g three times. The final concentrations of nanoparticles were established by UV-Vis absorbance spectroscopy (Shimadzu UV-2450). FIG. 2 is a transmission electron microscope (TEM) image of the MPdots; scale bar=20 nm.

The MPdots were conjugated with L-dihydroxyphenylacetic acid (L-DOPA), which is a conversion product of the amino acid, tyrosine and a substrate for the enzyme tyrosine hydroxylase that converts it into dopamine. L-DOPA is specifically taken up by dopaminergic and noradrenergic neurons for conversion to dopamine and norepinephrine in the respective neurons. In a typical conjugation of carboxyl groups on MPdots, 200 μL of HEPES buffer (1M, pH 7.4) were added to 10 mL of MPdots solution (0.5 mg/10 mL) prepared as described above. L-DOPA (1 mg; Sigma Aldrich, St. Louis, Mo.) was then added to the solution and mixed well. Next, freshly prepared N-ethyl-N′-(3-(dimethylamino propyl) carbodiimide (EDC 1%) and N-hydroxysuccinimide (NHS 1.5%) solution was added to the mixture. The mixture was magnetically stirred for 1 h at room temperature. The resulting MPdot conjugates were concentrated and purified with a 100 K Amicon® ultrafilter as described above.

Six vials containing 0.2 mL of 0.1, 1, 5, 10, 15 and 20 μg/mL MPdot solutions were imaged using a 7T Varian (Agilent) MR (magnetic resonance) imaging system. FIG. 3 shows the MR intensity of the particles.

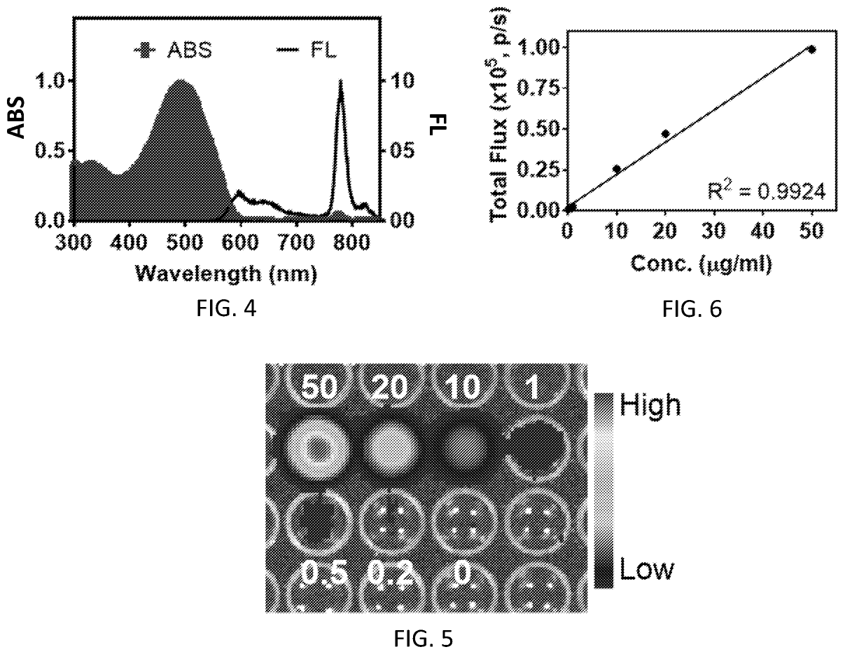

To determine the optical properties of the nanoparticles, the UV-Vis absorbance fluorescent emission spectra (λₑₓ=488 nm) of MPdots were measured (FIG. 4). Various concentrations of MPdots (0, 0.2, 0.5, 1, 10, 20, 50 μg/mL) were placed on a 96-well plate. The near-infrared afterglow (persistence luminescence) images were taken by irradiation with a white LED flashlight for 2 minutes. The images were taken on an IVIS Lumina II imaging system in the bioluminescence mode (FIG. 5). The exposure time was 5 minutes. The color scale bar represents the luminescence intensity in the unit of radiance, p/sec/cm2/sr. FIG. 6 shows the total flux as a function of concentration.

### Example 2

### Cell Viability

To determine whether the bimodal nanoparticles have any toxic effects, the effects of varying concentrations of the unconjugated MPdots on the viability of two different cells of neuronal origin were investigated: i) PC12 cells and ii) neural progenitor (NP) cells. PC12 and NP cells were grown and were exposed to varying concentrations of MPdots (0-50 μg/ml) for 24 h.

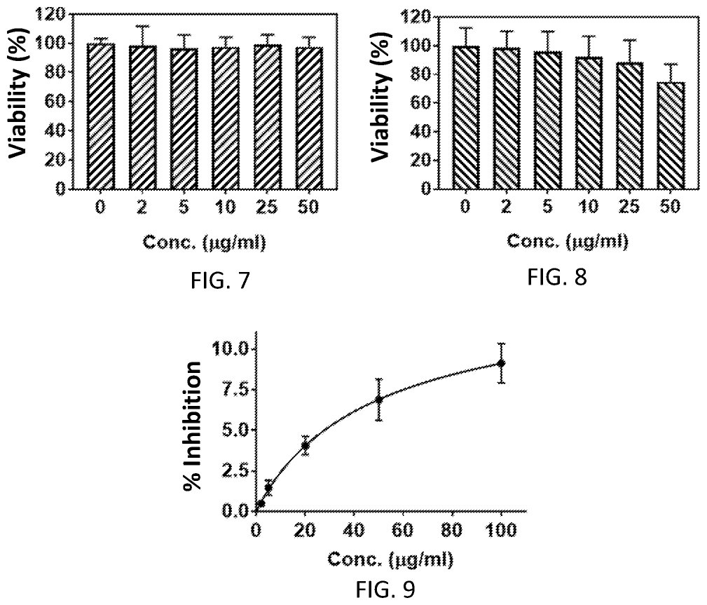

In the PC12 cell assay, the cells (1×10⁵/well) were added to the solution of MPdots with concentrations varying from 0 to 50 μg/mL, and co-incubated in a 24-well plate for 24 h in RPMI-1640 medium with 10% horse serum (heat inactivated), 5% fetal bovine serum (FBS), 50 U/mL penicillin, 50 μg/mL streptomycin and 250 ng/mL Amphotericin B. The cells were maintained in a humidified cell culture incubator at 37° C. under 5% CO₂. After 24 h, the incubated cells were counted using a hemocytometer with a 0.4% solution of trypan blue. The results are shown in FIG. 7.

In the neural progenitor cell assay, the cells (1×10⁵/well) were seeded in the solution of MPdots with concentrations varying from 0 to 50 μg/mL, and co-incubated in a 2% matrix gel (Corning)-coated 24-well plates for 24 h using AB2 basal neural medium (ArunA) supplemented with 2% ANS neural medium supplement (ArunA), 1% L-glutamine, 50 U/mL penicillin, 50 μg/mL streptomycin, 0.02 μg/mL bFGF (R&D Systems) and 0.01 μg/mL leukemia inhibitory factor (LIF, Millipore). The cells were maintained in a humidified cell culture incubator at 37° C. under 5% CO₂. After 24 h of incubation, the wells were replenished with AB2 basal neural medium. Then the incubated cells were counted using a hemocytometer with a 0.4% solution of trypan blue. The results are shown in FIG. 8.

The results of the assays show that incubation with even the highest concentration of MPdots (50 μg/mL) for 24 hours did not affect the cell viability (n=3).

Cell viability was also determined in an MTT (3-(4,5-dimethylthiazol-2-yl)-2,5-diphenyltetrazolium bromide) assay with PC12 cells. PC12 cells (1.5×10⁵) were seeded into a 96-well plate and maintained 24 h in 0.1 mL of the complete culture medium with MPdots at concentrations varying from 0 to 100 μg/mL. After 24 h incubation, 10 μL MTT reagents were added to each well and then incubated for 3 h at 37° C. (until purple precipitate was visible). 100 detergent reagents were added to each well. Absorbance was determined at 562 nm (n=3). The results are shown in FIG. 9. Even with the highest concentration of 100 μg/mL, the maximum inhibition observed was less than 10%.

### Example 3

### Imaging Brain Dopaminergic Activity

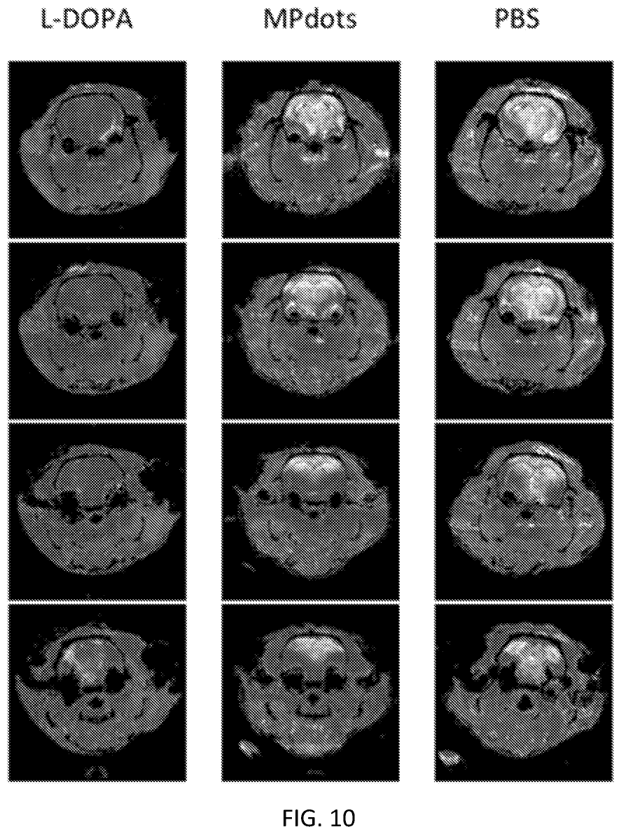

Adult Sprague-Dawley rats (4-5 month old) were administered intracerebroventricular (ICV) injections of vehicle (PBS), MPdots without L-DOPA (MPdots) or MPdots conjugated to L-DOPA (L-DOPA). The SD rats were anesthetized by isoflurane and then injected with 1 mg/Kg meloxicam. Under aseptic conditions, a stereotaxic instrument with nonpuncture ear bars (David Kopf Instruments) was used. For ICV injection, 26-gauge Hamilton syringes injected 5 μL of 2.6 mg/mL sample solutions (L-DOPA-MPdot, MPdots, and PBS buffer) to either the left or right lateral ventricle (from the bregma: −0.4 mm anteroposterior (AP), ±1.4 mm mediolateral (ML), −3.5 mm dorsoventral (DV) for rats). All phantom and in vivo MRI studies were performed on a 7T Varian (Agilent) MR imaging system using a 72-mm rat quadrature birdcage volume coil. The T2* mapping technique was performed with eight sets of spin echo images (the readout echo shifted 5 ms, 10 ms, 15 ms, 20 ms, 25 ms, 30 ms, 35 ms and 40 ms, respectively) and the following parameters: TR=1500 ms, field of view=40 mm×40 mm, scan matrix=256×256, slice thickness=1.00 mm, 10 slices, gap=0.20 mm. The results are shown in FIG. 10. The light areas in the middle (MPdots) and right (PBS) columns indicate an absence of MPdots in the brain. The darker areas in the images in the left column indicate the presence of L-DOPA-MPdot conjugates in the brain.

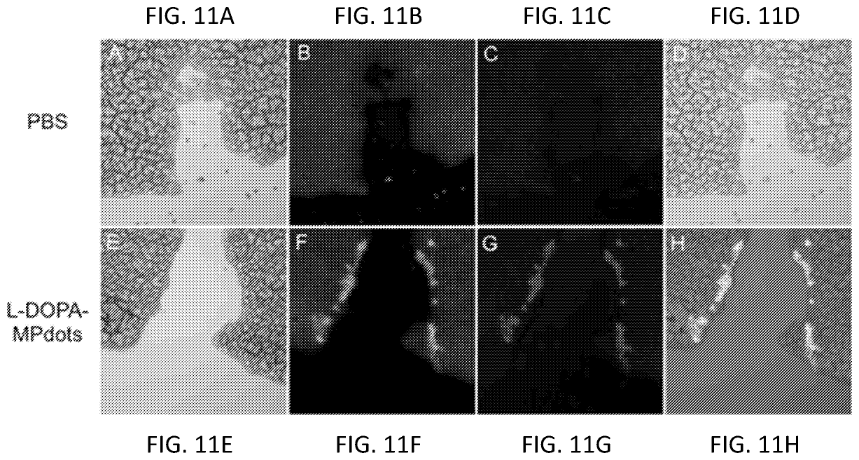

At the end of 72 h after MR imaging, animals were sacrificed and their brains were removed, sectioned and imaged for nanoparticles to determine the viability of bimodal imaging. The brains were collected and then frozen at −80° C. The frozen brains were sectioned as 25 μm thick in the cryostat. The brain sections were imaged on a Nikon MR confocal microscope. The results are shown in FIGS. 11A-H: FIGS. 11A-D are control brain sections; FIGS. 11E-H are L-DOPA-MPdot-labeled brain sections. The bright-field images were recorded in FIGS. 11A and 11E. The sections were then excited by 488 nm to obtain green fluorescent images (FIGS. 11B and 11F) and simultaneously obtain NIR fluorescent images (FIGS. 11C and 11G). The first three images in each row were overlapped to provide merged images (FIGS. 11D and 11H). The results show that perventricular dopaminergic neurons are labeled with ICV injections of L-DOPA MPdots. In contrast, ICV injection of PBS did not label any cells.

The foregoing results provided evidence that the bimodal nanoparticle conjugates successfully target dopaminergic neurons and can be used to image dopaminergic neurons.

However, ICV administration was used to obtain the data. Non-invasive administration of MPdots to image neurotransmitters is important for use in a clinical setting. Thus, intravenous administration was investigated to determine whether the bimodal nanoparticle conjugates could cross the blood-brain barrier and reach dopaminergic neurons.

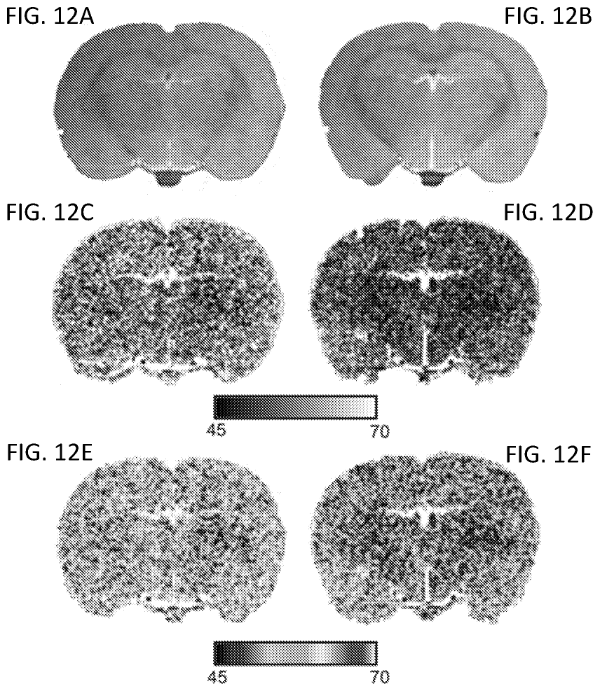

Adult SD rats were injected intravenously via the jugular vein with either PBS (vehicle) or L-DOPA-MPdot conjugates. The animals were imaged using a 7T Varian (Agilent) MR imaging system using 72 mm rat quadrature birdcage volume coil 72 h after the injection. The results are shown in FIGS. 12A-F. FIGS. 12A, 12C, and 12E are brain sections from animals injected with PBS; FIGS. 12B, 12D, and 12F are images from animals injected with the L-DOPA-MPdot conjugates. FIGS. 12A and 12B are qualitative T2-weighted images; FIGS. 12C and 12D are quantitative T2 maps (the gray scale units are milliseconds); FIGS. 12E and 12F are quantitative T2 maps with a pseudo color scale (the color scale units are milliseconds). The darker image of FIG. 12D compared to FIG. 12C indicates that the L-DOPA-MPdot conjugates successfully crossed the blood-brain barrier. Likewise, the increased blue color of FIG. 12F compared to FIG. 12E also indicates that the L-DOPA-MPdot conjugates successfully crossed the blood-brain barrier.

The luminosity after PBS and L-DOPA-MPdot conjugate administration was quantified in areas of interest. 2D-reconstruction of coronal rat brain sections was performed as described The Rat Brain in Stereotaxic Coordinates (Paxinos and Watson, 7^{th} edition, 2013). MR images were matched with the reconstructed coronal sections and T2 map values from FIGS. 12C and 12D were calculated using the luminosity option. The results are shown in Table 1.

TABLE 1

Luminosity (T2 Map

| | Left CPu | Right CPu |
|---|---|---|
| PBS | 129.42 (57.64 ms) | 119.12 (56.63 ms) |
| L-DOPA-MPdot | 103.86 (55.14 ms) | 86.06 (53.40 ms) |

### Example 4

### Brain Imaging after Intravenous Administration

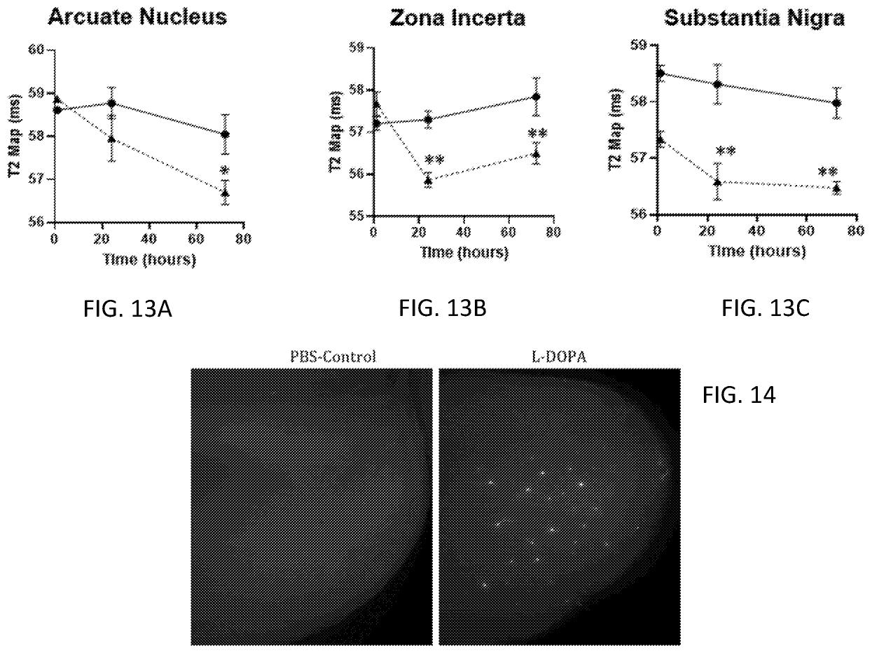

Sprague Dawley (SD) rats were given vehicle (200 μl PBS) or L-DOPA MPdot conjugates (in 200 μl PBS) i.v. through the jugular vein under anesthesia. Quantitative T2 mapping (MRI) was obtained at three time points: 1 h, 24 h and 72 h after i.v. injections; qualitative T2-weighted maps of the regions of interest (ROIs) (arcuate nucleus, zona incerta and substantia nigra) were obtained. These three areas are characteristic dopaminergic areas. The selected T2 map images were converted by Mango (V4.1) to 8-bit gray PNG files with specifically normalized T2 map range. A standard coronal sections of rat brain was obtained from The Rat Brain in Stereotaxic Coordinates (George Paxinos, Georgia & Watson, Charles. 7^{th} edition. Academic Press, 2014). The matched brain diagrams were processed in Adobe® Illustrator® 2018 software to get region patterns in brain slides. The T2 map images were matched to the 2D reconstruction brain diagrams in Adobe® Photoshop® 2018 CC software. Mean values of each ROI were obtained by using a Histogram's luminosity option. The real values of T2 map were calculated based on luminosity, scale ranges and resolutions of images. Finally, the T2 map data from different ROIs was analyzed by repeated measures ANOVA using GraphPad Prism (V8.2.1). FIGS. 13A-13C show the T2 map data for the arcuate nucleus (13A), zona incerta (13B), and substantia nigra (13C); * p<0.05, ** p, 0.01, repeated measures ANOVA. Lower numbers correlate with higher intensity. In each case, the L-DOPA-MPdot conjugate (▴) shows higher intensity than the vehicle (●).

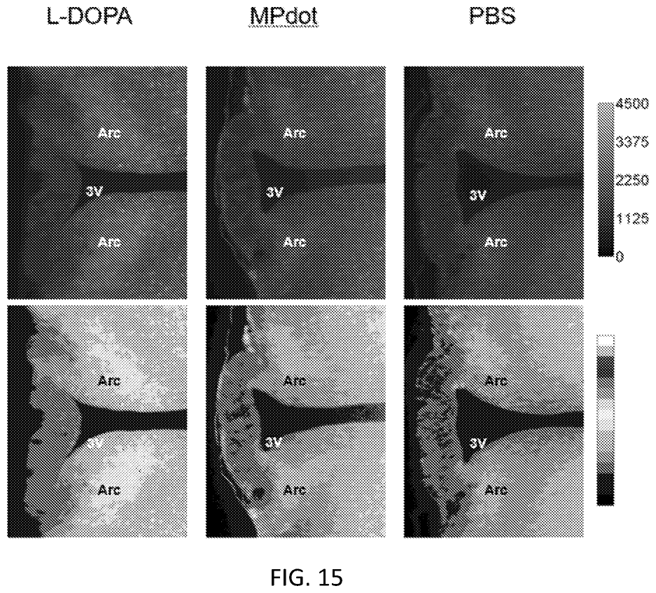

Female Sprague Dawley (SD) rats were given vehicle (200 μl PBS) or L-DOPA MPdot conjugates (in 200 μl PBS) i.v. through the jugular vein under anesthesia. Two hours later, rats were perfused with 10% paraformaldehyde under anesthesia. Multiple organs including the liver, lung, spleen and the brain were removed and were cut to fit sample holders. The organs were cleared using immunolabeling-enabled imaging of solvent-cleared organs (iDISCO—immunolabeling-enabled three-dimensional imaging of solvent-cleared organs) protocol. Samples were initially dehydrated by methanol (MeOH), lipids were extracted using incubation with dichlormethane (DCM), and then immersed in dibenzyl ether (DBE) for refractive index (RI) matching. Finally, samples were imaged using a light sheet microscope (UltraMicroscope II light sheet microscope, LaVision BioTec). The samples were irradiated by 488 nm laser for excitation of MPdots and then emission light was collected between 500 nm and 550 nm. The images were recorded by a 4 X objective lens. FIGS. 14A and 14B show representative images of hindbrain from an animal injected with vehicle (PBS control) or L-DOPA MPdots (L-DOPA).

Female Sprague Dawley (SD) rats were given vehicle (200 μl PBS), MPdots without L-DOPA, or L-DOPA MPdot conjugates (in 200 μl PBS) i.v. through the jugular vein under anesthesia. Two hours later, the rats were perfused with 10% paraformaldehyde under anesthesia. Multiple organs including the liver, lung, spleen and the brain were removed and were cut to fit sample holders. The organs were cleared using immunolabeling-enabled imaging of solvent-cleared organs (iDISCO) protocol. Samples were initially dehydrated by methanol (MeOH), lipids were extracted using incubation with dichlormethane (DCM), and then immersed in dibenzyl ether (DBE) for refractive index (RI) matching. Finally, samples were imaged using a light sheet microscope (UltraMicroscope II, LaVision BioTec). The samples were irradiated by 488 nm laser for excitation of MPdots and then emission light was collected between 500 nm and 550 nm. The images were recorded by a 2.5 X objective lens. The raw data was converted to either green color scale (top three panels) or 16-color scale (bottom three panels). FIG. 15 shows representative images (in grayscale) of the acruate nucleus from animals injected with L-DOPA MPdots (L-DOPA), unconjugated MPdots (MPdot), or the vehicle (PBS); Arc=arcuate nucleus, 3V=3^{rd} ventricle.

### Example 5

### Non-Invasive Imaging

A subject in need of central nervous system tissue imaging is identified. The subject may be identified on the basis of laboratory testing of evaluation by a clinician.

The subject is administered a pharmaceutical composition comprising ligand-bimodal nanoparticle conjugates as disclosed herein. The subject is administered with an amount of the pharmaceutical composition effective for subsequent imaging, e.g., an amount of the pharmaceutical composition sufficient to provide 100-500 μg/kg body weight of the ligand-bimodal nanoparticle conjugates. The pharmaceutical composition may be administered by intravenous injection. After a suitable period of time (e.g., from an hour to ten days), at least a portion of the subject's central nervous system tissue is imaged. For example, at least a portion of the subject's brain tissue or spinal cord tissue may be imaged. Imaging may include magnetic resonance imaging, fluorescence resonance energy transfer imaging, or a combination thereof.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A ligand-bimodal nanoparticle conjugate, comprising:
a bimodal nanoparticle comprising
a polymeric matrix,
one or more magnetic particles disposed within the polymeric matrix or conjugated to an outer surface of the polymeric matrix, and
a near-infrared dye disposed within the polymeric matrix; and
a ligand for a blood-brain barrier amino acid transporter, the ligand conjugated to the outer surface of the bimodal nanoparticle.

2. The ligand-bimodal nanoparticle conjugate of claim 1, wherein the ligand comprises an amino acid precursor of a neurotransmitter.

3. The ligand-bimodal nanoparticle conjugate of claim 1, wherein the ligand comprises levodopa (L-DOPA), 5-hydroxytryptophan (5-HTP), or a combination thereof.

4. The ligand-bimodal nanoparticle conjugate of claim 1, wherein the one or more magnetic particles comprises a magnetic metal, a compound comprising a magnetic metal, or a combination thereof.

5. The ligand-bimodal nanoparticle conjugate of claim 4, wherein the one or more magnetic particles comprises Fe₃O₄, Fe₂O₃, Fe, Gd, or a combination thereof.

6. The ligand-bimodal nanoparticle conjugate of claim 1, wherein the near-infrared dye comprises silicon 2,3-naphthalocyanine bis(trihexylsilyloxide) (NIR775).

7. The ligand-bimodal nanoparticle conjugate of claim 1, wherein the polymeric matrix comprises a semiconducting polymer and an amphiphilic polymer.

8. The ligand-bimodal nanoparticle conjugate of claim 7, wherein the polymeric matrix comprises:
poly[2-methoxy-5-(2-ethylhexyloxy)-1,4-phenylenevinylene](MEH-PPV), poly[2,5-bis(octyloxy)-1,4-phenylenevinylene](BOPPV), poly(5-(2-ethylhexyloxy)-2-methoxycyanoterephthalylidene) (MEHCPV), poly[2-methoxy-5-(2-ethylhexyloxy-1,4-phenylene]end-capped with dimethylphenyl (MEHPP), poly[2-methoxy-5-(3′,7′-dimethyloctyloxy)-1,4-phenylenevinylene](MDMO-PPV), poly[9,9-dioctylfluorenyl-2,7-diyl)-alt-co-(1,4-benzo-(2,1′,3)-thiadiazole)](PFBT), poly(2,5-dioctyl-1,4-phenylenevinylene) (POPPV), or any combination thereof; and
polystyrene grafted with carboxyl-group-functionalized ethylene oxide (PS:PEG:COOH), poly(ethylene glycol)-block-polylactide (PEG-PLA), poly(ethylene glycol)-block-poly(lactide-co-glycolide) (PEG-PLGA), poly(ethylene glycol)-block-polyethylene (PEG-PE), poly(ethylene glycol)-block-poly(ε-caprolactone) (PEG-PCL), poly(ethylene glycol)-block-poly(propylene glycol)-block-poly(ethylene glycol) (PEG-PPG-PEG), poly(propylene glycol)-block-poly(ethylene glycol)-block-poly(propylene glycol) (PPG-PEG-PPG), or any combination thereof.

9. The ligand-bimodal nanoparticle conjugate of claim 8, wherein the polymeric matrix comprises MEH-PPV and PS:PEG:COOH.

10. The ligand-bimodal nanoparticle conjugate of claim 9, wherein the polymeric matrix comprises MEH-PPV and PS:PEG:COOH in a weight ratio of from 1.5:1 to 1:1.5.

11. The ligand-bimodal nanoparticle conjugate of claim 1, wherein:
(i) a diameter of the ligand-bimodal nanoparticle conjugate is ≤100 nm; or
(ii) the ligand-bimodal nanoparticle conjugate has a ligand:bimodal nanoparticle weight ratio of from 0.5:1 to 10:1; or
(iii) both (i) and (ii).

12. (canceled)

13. The ligand-bimodal nanoparticle conjugate of claim 1, comprising:
a bimodal nanoparticle comprising
a polymeric matrix comprising MEH-PPV and PS:PEG:COOH,
one or more Fe₃O₄ particles disposed within the polymeric matrix, and
NIR775 disposed within the polymeric matrix; and
L-DOPA conjugated to an outer surface of the bimodal nanoparticle.

14. A pharmaceutical composition comprising:
a plurality of ligand-bimodal nanoparticle conjugates according to claim 1; and
a pharmaceutically acceptable carrier.

15. A method, comprising:
administering a plurality of ligand-bimodal nanoparticle conjugates according to claim 1 to a subject; and
subsequently imaging central nervous system tissue in the subject.

16. The method of claim 15, wherein imaging the central nervous system tissue in the subject comprises magnetic resonance imaging, fluorescence resonance energy transfer imaging, or a combination thereof.

17. The method of claim 15, wherein the ligand-bimodal nanoparticle conjugates comprise L-DOPA, and imaging the central nervous system tissue in the subject visualizes dopaminergic neurons, noradrenergic neurons, or both dopaminergic and noradrenergic neurons bound by the ligand-bimodal nanoparticle conjugates.

18. The method of claim 15, wherein the ligand-bimodal nanoparticle conjugates comprise 5-HTP, and imaging the central nervous system tissue in the subject visualizes serotonergic neurons bound by the ligand-bimodal nanoparticle conjugates.

19. The method of claim 15, wherein administering the ligand-bimodal nanoparticle conjugates comprises intravenously injecting the ligand-bimodal nanoparticle conjugates or a pharmaceutical composition comprising the ligand-bimodal nanoparticle conjugates.

20. The method of claim 15, wherein the subject is a human.

21. The method of claim 15, wherein:
(i) administering the ligand-bimodal nanoparticle conjugates comprises administering from 100-500 μg of the ligand-bimodal nanoparticle conjugates per kilogram body weight to the subject; or
(ii) imaging is performed from one hour to ten days after administering the plurality of ligand-bimodal nanoparticle conjugates to the subject; or
(iii) both (i) and (ii).

22. (canceled)
